(19) United States
(12) Patent Application Publication
(10) Pub. No.: US 2009/0118684 A1
(43) Pub. Date: May 7, 2009

(54) THERMAL PERSONAL CARE SYSTEMS AND METHODS

(76) Inventors: Luiz B. DA SILVA, Danville, CA (US); T. Debuene CHANG, Danville, CA (US); Igor G. KOCHEMASOV, Nizhny Novgorod (RU)

Correspondence Address: LEVINE BAGADE HAN LLP, 2483 EAST BAYSHORE ROAD, SUITE 100, PALO ALTO, CA, 94303, US

(21) Appl. No.: 12/263,159

(22) Filed: Oct. 31, 2008

Related U.S. Application Data
(60) Provisional application No. 61/002,088, filed on Nov. 5, 2007.

(51) Int. Cl. A61M 35/00; A61K 8/00; A61F 7/00; A61B 17/50

(52) U.S. Cl. 604/290; 510/130; 604/291; 604/310; 607/104; 607/109; 606/131

(57) ABSTRACT

Devices and methods for the treatment of skin conditions and lesions are disclosed herein. A thermal device can be used to heat or cool a personal care pack that can then be used to cleanse, treat, and rejuvenate the skin. The personal care pack can be formulated with smooth or abrasive material and can contain a variety of liquids, lotions or gels. Application of the personal care pack, at the desired temperature to the skin can stimulate deeper blood circulation, clear pores, diminishes or removes deep and fine wrinkles, accelerate acne clearing, treat blemished skin, decrease edema and erythema of the skin, and reduce puritis of various etiologies.

### CROSS-REFERENCE TO RELATED APPLICATIONS

This application claims priority to U.S. Provisional Application No. 61/002,088, filed Nov. 5, 2007, which is incorporated by reference herein in its entirety.

### BACKGROUND OF THE INVENTION

A variety of devices and lotions are available to cleanse and rejuvenate skin. Methods of rejuvenating skin range from aggressive techniques such as face lift surgery, laser skin resurfacing, and chemical peel to milder techniques such as non-ablative lasers systems, RF energy skin rejuvenation systems, and micro-dermabrasion. These techniques offer the potential for a younger looking appearance, the removal of wrinkles, clearing pores, reducing pitting and scarring regions, the reduction or removal of discolorations and darkened areas of the skin, and the possibility of clearing some types of undesirable skin conditions (e.g. acne, scars, and rosacea). Most of these systems are large and require professional treatment.

Smaller handheld devices with abrasive tips (e.g., U.S. Pat. No. 2,712,823 and U.S. Pat. No. 4,378,804) have also been previously described for use in rejuvenating skin and removing skin blemishes. In previously described cases, the treatment tip was at room or skin temperature. The use of hot or cold treatment pads or device has the potential to stimulate deeper blood flow, reduce pore size, and also reduce discomfort during treatment. A need exists for a handheld hot or cold applicator that can effectively cleanse and rejuvenate skin, hair, or a combination thereof.

### SUMMARY OF THE INVENTION

Personal care systems and methods for rejuvenating skin, treating skin conditions and skin lesions such as acne, removing fine wrinkles, preventing discoloration and blemishes, reducing edema and swelling, calming dermatitis and skin irritations, and clearing skin of other inflammatory and erythematosus processes are disclosed. A hand held device that can be safely used to exfoliate and cleanse skin with minimal discomfort or pain while leaving a moisturizing and protective layer for longer term treatment is also disclosed. A device that can heat or cool personal care products or materials to increase or accelerate efficacy is also disclosed. The personal care products may include soaps, creams, wipes or towelettes, washes, shampoos, conditioners, sprays, lotions, balms, enamels, tonics, mousses or combinations thereof. The personal care products can be applied, and designed for application, on the face, hands, body, hair, eyes, lips, or combinations thereof.

A thermal device that can cool and/or heat an encapsulated personal care product pack and treatment hand piece are disclosed. The encapsulated personal care product pack can be placed within the thermal device and cooled or heated to a desired temperature. The encapsulated pack can be substantially thermally insulated. The encapsulated pack can have pack contents, such as a fluid or gel or cream. The encapsulated pack can be a cartridge, blister pack, ampoule, or other openable container. The encapsulated pack can have a removable protective cover that can contain the pack contents in the pack. The thermal device can cool the pack can low enough to freeze the pack contents. The thermal device can heat the pack enough to vaporize the pack contents. When sufficiently heated or cooled (e.g., with frozen pack contents), the pack can be removed from the thermal device and the protective cover can be removed from the pack. The pack can subsequent to, concurrent with, or predicate to removal of the protective cover, the pack can be attached to the hand piece. The hand piece can be manipulated to apply the pack and/or the pack contents to the skin. Rubbing the skin with the pack can exfoliate the skin, for example, by removing the top layers of the stratum corneum and cleansing the skin of dirt, grease, and debris. A hot temperature of the pack can stimulate blood flow in the dermis. The cold temperature of the pack can stimulate blood flow in the dermis as a reflexive response to cold in the upper surface layers of the epidermis, reduce inflammation, edema with minimal discomfort or pain, even when more aggressive dermabrasion is performed.

The pack contents can include drugs or pharmaceuticals (e.g. benzoyl peroxide, salicylic acid, glycolic acid, antiseptic, antibiotics, steroids, anti-inflammatories) and/or cosmetics (e.g. emollients, moisturizers, botanicals, fragrances, vitamins, and anti-oxidants), for example, that are effective at treating a variety of skin conditions. Through the use of the applicator, these pharmaceuticals and cosmetics can be left on the skin surface after application to the skin, for example to sooth, moisturize, and protect the skin for long term treatment afterwards.

The pack can also include an abrasive fluid absorbing layer that can be used to aggressively remove the upper keratinized cells layers from the stratum corneum of the epidermis, allowing for easier and more effective absorption of pharmaceuticals and cosmetics and can simultaneously leave a surface layer of protective or soothing moisturizer or emollient.

Before the hand piece is applied to the skin, for example, the thermal device can be preloaded and programmed to heat or cool and prepare the pack for use at a selected and convenient time for personal use. Cooling or heating of the cold pack can take less then about 20 minutes, or for example less than about 5 minutes.

The encapsulated pack can be placed within the thermal device and warmed to a desired temperature which is, for example between about 25° C. and about 100° C., or more narrowly between about 40° C. and about 80° C. When warmed to the desired temperature, the pack can be removed from the thermal device. A protective cover on the pack can then be removed, or the pack can be otherwise opened, before, concurrent with, or subsequent to loading (e.g., attaching to or inserting in) the pack to a handpiece.

The handpiece can then be used to apply the warm pack, and/or the pack contents, to the skin. Rubbing the skin with the warmed or cooled pack can exfoliate the skin, for example removing the top layers of the stratum corneum and cleansing the skin of dirt, grease, and debris. The warm temperature of the pack can stimulate blood flow, open pores to expose dirt, debris, and bacteria to deeper cleansing. Warm cleansing can also allow more delivery of humoral and blood factors to clear lesions and resolve edema and erythema. The fluid, gel, lotion, or cream contained within the warm pack can include drugs or pharmaceuticals (e.g. benzoyl peroxide, salicylic acid, glycolic acid, antiseptic, antibiotics, steroids, anti-inflammatories) and cosmetics (e.g. emollients, moisturizers, botanicals, fragrances, vitamins, and anti-oxidants) which are effective at treating a variety of skin conditions. Through the use of the applicator, these pharmaceuticals and cosmetics can be left on the skin surface after treatment for soothing moisturization and protection for long term treatment afterwards.

The pack can have an abrasive fluid absorbing layer on the surface of the pack that can be used to aggressively remove the upper keratinized cells layers from the stratum corneum of the epidermis. For example, the abrading can encourage easier and more effective absorption of pharmaceuticals and cosmetics by the skin and can simultaneously leave a surface layer of protective or soothing moisturizer or emollient.

The thermal device can have multiple thermal functions. For example, the thermal device can cool and/or heat the personal care product pack. The personal care product pack can be coded (or keyed) so that the thermal device can automatically determine the final desired temperature and the thermal device heats or cools as appropriate. The user can manually select the operating mode of the thermal device, for example through a control such as a switch or button.

### BRIEF DESCRIPTION OF THE DRAWINGS

FIG. 1 illustrates a variation of the personal care system.

FIG. 2 illustrates a variation of a method for using a variation of the disposable personal care packs to cleanse and treat the face.

FIG. 3 illustrates a sectional view of a variation of the disposable personal care pack during use.

FIGS. 4 and 5 illustrate sectional views of variations of the disposable personal care pack container for maintaining and storing the disposable personal care pack before use.

FIG. 6 illustrates a sectional view taken through one variation of the thermal device.

FIG. 7 illustrates a schematic diagram of a variation of the thermal device.

FIG. 8 illustrates a variation of a method for attaching the personal care pack to the treatment handle.

FIG. 9 illustrates a variation of the personal care system, for example without the need for disposable personal care packs.

### DETAILED DESCRIPTION

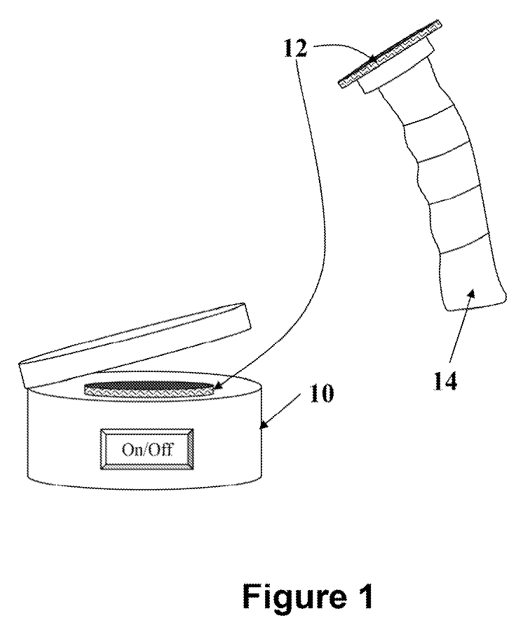

personal care skin treatment system that can be safely used by adolescents and adults to cleanse, rejuvenate and treat skin conditions such as acne, blemished skin, dermatitis including eczema, hives, or other inflammatory and/or edematous lesions such as rosacea, or fine wrinkles is described herein. FIG. 1 illustrates that the personal care skin treatment system can have a thermal device 10, a disposable personal care pack 12, and hand piece 14. The personal care skin treatment system can be used to the apply the personal care pack 12, and/or the pack contents inside the personal care pack 12, to the skin.

The personal care pack 12 can be cooled or heated by the thermal device 10 before the application of the personal care pack 12 to the skin, for example to reduce pain during micro-abrasion, skin tightening due to the cooling of skin which reduces pore size and stimulated 12 deeper blood circulation in the dermis to help carry humoral, immune, and blood factors to the affected regions and speed healing. Use of the heated personal care pack can increase blood circulation, accelerate chemical and enzymatic reactions, increase penetration of personal care product through the skin, open pores to expose dirt, debris, and bacteria to deeper cleansing, and combinations thereof. The efficacy of personal care products can be directly increased by the elevated temperature which can accelerate chemical reactions.

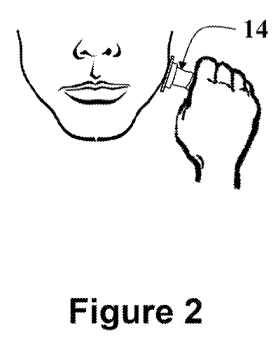

FIG. 2 shows how the system can be used to treat skin on the face. The hand piece 14 with an attached personal care pack 12 can be placed on the skin to treat. The hand piece 14 can be not powered. The user can rub the personal care pack on the skin by moving the personal care pack 12 in a circular or back and forth pattern over the skin area to treat.

The hand piece 14 can be powered. The hand piece 14 can rotate and/or vibrate the personal care pack 12 automatically. The user can slide the personal care pack on the region of the skin to be treated. Each personal care pack can be disposable. A new heated or cooled personal care pack can be loaded on the hand piece 14 for each use. A given skin area can be treated once or twice a day with a hot and/or cold personal care pack.

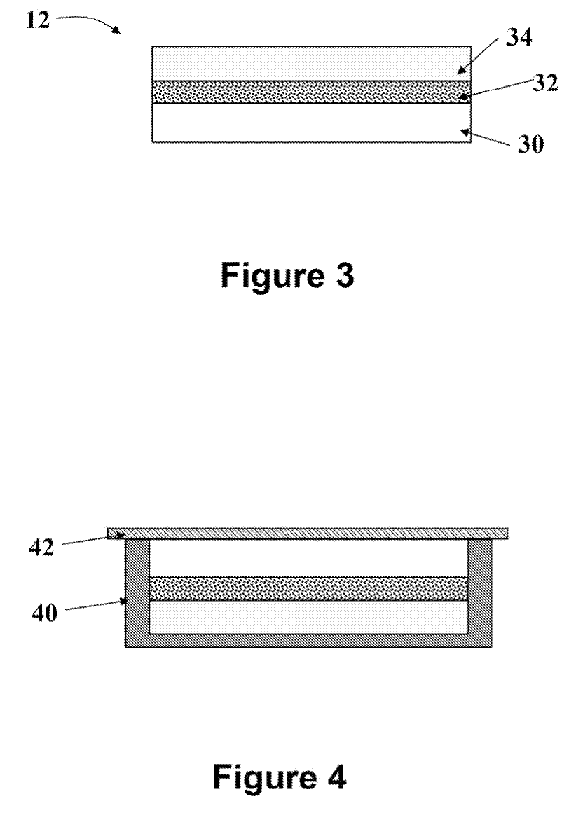

FIG. 3 shows that the personal care pack 12 can have about three distinct layers. The layers can include a hard plastic support 30, a fluid absorbing layer 32 and fluid layer 34. The hard plastic support 30 can attach firmly to the hand piece 14 so that the personal care pack 12 can be easily applied to skin without the user touching the personal care pack 12. The fluid absorbing layer 32 can either be soft (e.g., tissue, cloth, foam), hard, smooth, abrasive (structured plastic, porous ceramic, porous metal), or combinations thereof. The fluid layer 34 can penetrate into the fluid absorbing layer 32 and can have a wide variety of gels, creams, liquids with and without pharmaceuticals and cosmetics (e.g., water, anti-oxidants, antibiotics, anti-inflammatories, moisturizers, emollients, vitamins, fragrance, and cleansing and abrasive elements). The fluid layer 34 can be frozen by the thermal device before the personal care pack 12 is applied to skin and melts as the personal care pack 12 is rubbed on the skin to abrade, debride, clean the skin, and leave a residual layer of moisturizer and protectant. The fluid layer 34 can be cooled, for example but left unfrozen, before application to the skin. The personal care pack 12 can be heated before application to the skin.

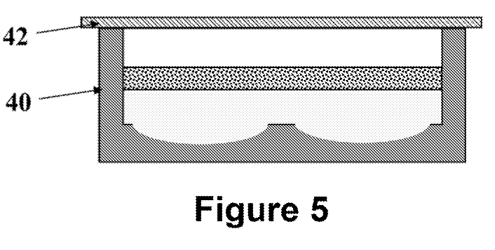

FIG. 4 shows that the personal care pack 12 can be contained during storage and before application to the skin. The personal care pack 12 can be contained within a package that can have a hard plastic shell 40 with a flat bottom surface. An easily removed thin cover 42 can cover an open side of the hard plastic shell 40. The packaging of the personal care pack 12 can be leak-proof sealed, for example preventing the fluid layer from leaking out.

FIG. 5 shows that the personal care pack 12 can be contained during storage and before use. The hard plastic shell 40 can be structured to contour the fluid layer to the desired shape. The structures could be smooth as shown in FIG. 5 or rough to provide a more abrasive surface when frozen.

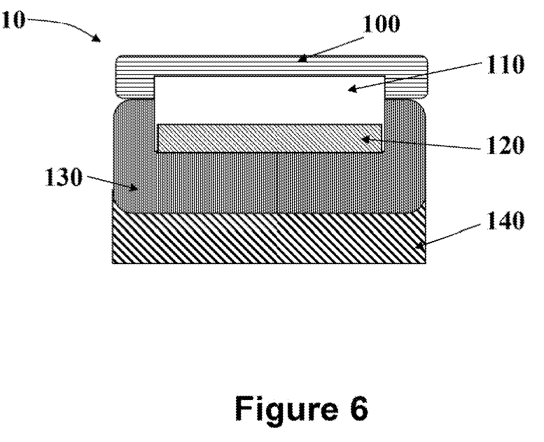

FIG. 6 shows a cross section of the thermal device 10 that can be used to heat or cool the personal care pack 12. The thermal device can have a cover 100 that can be opened to expose a volume 110. The volume 110 can be filled with a personal care pack 12. A thermoelectric module 120 within the thermal device can be used to heat or cool the personal care pack 12. This design can take advantage of the properties of thermoelectric modules in which one side can be heated or cooled depending in which direction current flows. The direction of current flow to the thermoelectric module 120 can be reversed, for example by a control on the thermal device 10.

The thermoelectric module 120 can connect to a heat sink 130 and power unit 140 that can control operation (e.g., temperature, on/off status, whether in heating or cooling mode) of the thermoelectric module 120. The thermoelectric module can be used to cool and/or freeze the personal care pack 12 when the personal care pack 12 is placed inside the thermal/cold device 10 to a temperature of less than about 10° C., or for example less than about 0° C. The thermoelectric module 120 can be used to heat the personal care pack 12 when the personal care pack 12 is placed inside the thermal device 10 to a temperature greater than about 27° C. and less than about 100° C., or more narrowly from about 35° C. to about 80° C., for example to minimize the risk of burning skin.

The personal care product 12 can be inserted into the thermal device 10, and cooling or heating can be initiated as desired. When the personal care pack 12 reaches the desired temperature, the user can be notified (e.g., beeping, and/or flashing lights) so that the personal care pack can be extracted and attached to the hand piece 14 for treatment. The time to heat or cool the personal care pack 12 can be less than about 20 minutes, or less than about 5 minutes. The personal care pack 12 can be coded (e.g. mechanical shape, RFID tag, bar code), for example to allow the thermal device 10 to automatically detect the desired or optimum temperature for the personal care pack 12.

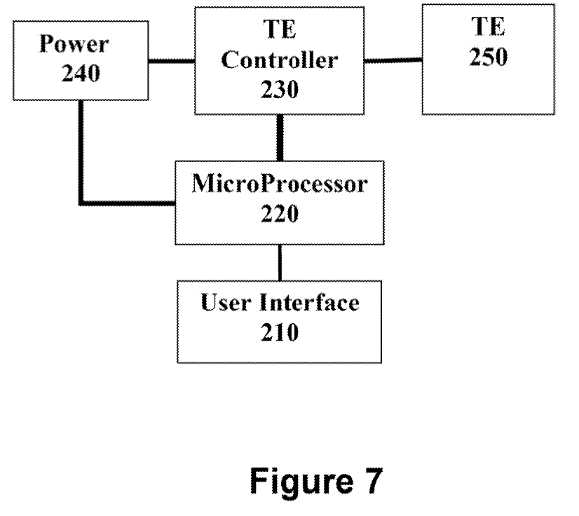

FIG. 7 shows a block diagram of the key elements of the thermal device 12. A power source 240 which can be a wall plug or batteries can power the thermoelectric module and controller 230, microprocessor 220, and user interface 210. The microprocessor 220 can accept user input (e.g. on/off, desired temperature, time to turn on and prepare) that can control device operation. User interface 210 can include push buttons, LCDs, LEDs and buzzers.

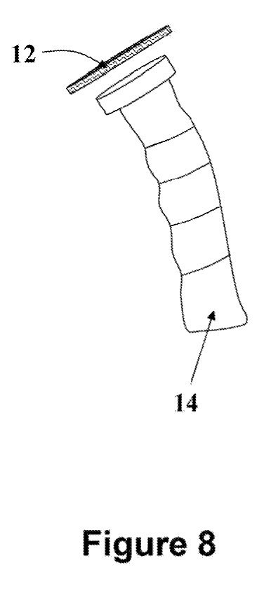

FIG. 8 shows how the cold pack 12 and hand piece 14 separate/attach. The hand piece 14 can be not powered and the hard plastic support layer 30 of the cold pack 12 simply snaps together with the hand piece 14. The hand piece can include a release mechanism that automatically releases the cold pack 12 after use. The hand piece can be battery powered and can have a motor to rotate or vibrate the cold pack 12 during use.

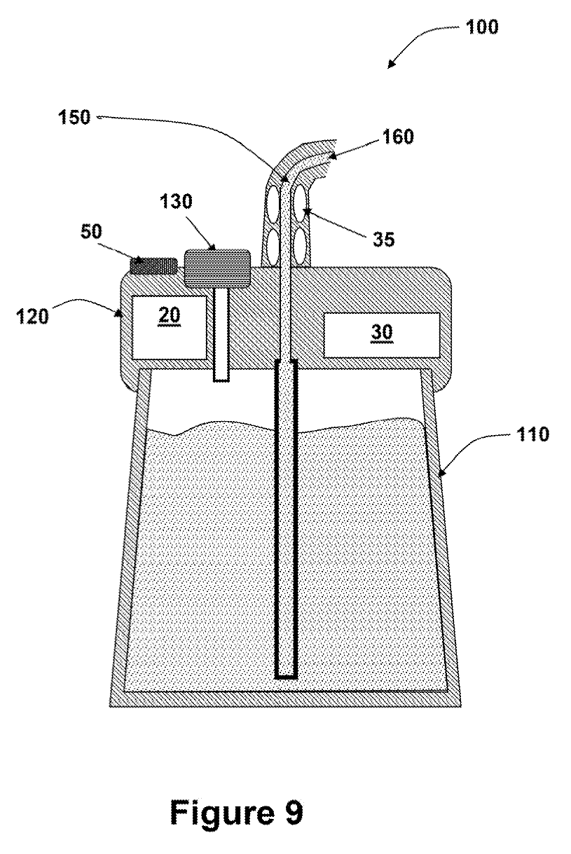

FIG. 9 shows that the system can have an automatic dispenser with a small thermal section. The device contains a power source 20 which can be batteries or a suitable power supply that can plug into a standard AC outlet. Control electronics 30 can accept user input and control operation of the device. User input can be a simple On/Off button or touch screen interface. Large volume liquid or lotion personal care products can be conveniently dispensed and prepared in smaller, individual single use amounts. The device 100 can have a large volume container 110 and a multi-use dispenser 120 that can attach to the container. The dispenser can contain a pumping mechanism 130 that can force the liquid or lotion from the container 110 through the heating or cooling section 150 before exiting out the dispensing tip 160. The thermal section 150 can use a thermoelectric module 35 that can heat or cool the personal care product. The thermal section 150 can heat the personal care product only. Heating can be accomplished by using resistive heating to heat the thermal section 150 to the desired constant temperature. Using a constant temperature thermal section can insure the personal care product is not dispensed at a high temperature that can cause burns but requires a small delay before material can be dispensed. When using this thermal device 100 the user simply presses the dispense button 50 and releases the button 50 when enough product has been dispensed.

The automatic pumping mechanism can be replaced by a user activated pumping mechanism. The thermal section 150 can automatically turn on when the user actuates the pumping mechanism.

The pumping mechanism can be eliminated and gravity can drive the liquid through the activation chamber. A push button or dial can turn on or off flow. The dispenser 120 can be on the bottom of the assembly and a fastener or hook on the container 110 can allow the user to hang the assembly.

Skin improvement effects by the methods of using the system can include, for example by cooling at the surface and allowing the energy to cool the upper layer of the dermis to achieve controlled changes to the collagen in the upper dermal layer. The system can temporarily tightening the skin from the cold and reducing the pore size of skin for a smoother texture of the skin. The system can, by heating the skin surface, induce controlled changes to the collagen in the upper dermal layer. The system can, by temporarily dilating skin pores with heat, allow deeper cleaning of dirt, debris, oils, contaminants, and bacteria. The system can deliver a controlled amount of mechanical abrasion to remove upper layers of the stratum cornium of the epidermis, resulting in easier access to pores and mechanical cleansing and debridement of dirt, contaminants, and excess sebum and bacteria. The system can allow more direct delivery of pharmaceuticals and cosmetics into deeper layers of skin for effect and treatment once the cold mechanical abrasion of the upper layers is accomplished. The system can decreasing edema and erythema of the skin thereby reducing post inflammatory hyper pigmentation preventing discoloration, uneven coloration, and blotching of the skin.

### Method of Use

The devices described herein are suitable for use in cleansing skin, rejuvenating skin and treatment of various skin conditions and lesions. These devices offer the potential for younger looking appearance; remove wrinkles, clearing of pores, reducing inflammation and edema, and the possibility of clearing some types of undesirable skin conditions (e.g. acne, rosacea, eczema, and other types of dermatitis).

The devices described herein are suitable for use in the treatment of various skin conditions and lesions. Examples of such skin conditions and lesions are provided herein, but are not limited to the conditions and lesions described herein. Bacterial infections lead to common inflammatory and edematous lesions such as acne and pimples. Other dermatitis lesions are caused by irritants, which may be introduced as a result of bug bites or by exposure to other natural or man-made skin irritants. Eczema can be widespread or localized on the skin and is consistent with inflammatory lesions. Some infections (either bacterial—. i.e. staphylococcus. Streptococcus, Pseudomonas or fungal—i.e. Candida in difficult to access areas such as paronychial infections and under the nail bed might be treated with pharmaceutical instilled dermabrasion

Other skin conditions include pustular eruptions, localized abscessed formation and local inflammatory conditions of the dermis and epidermis. One of the most common afflictions of this type is lesions caused by the condition known as acne vulgaris. Acne vulgaris is associated with the Gram-positive anaerobic bacterium, Propionibacterium acnes.

Eczema is a form of inflammatory lesions which can be localized or widespread and may be a response to known or unknown irritants. Eczema is puritic and irritating. Long-term, chronic inflammation and edema can lead to post inflammatory hyper pigmentation and discoloration of the skin with unsightly, uneven blotchiness.

Methods and devices for the treatment of skin conditions and skin lesions involving the application of mechanical abrasion and cooling and/or gentle heating to the infected or irritated tissue are disclosed. The system alone or in combination with other pharmaceuticals (e.g. antifungal, antiviral) can be used to treat skin lesions caused by bacterial, fungal, or viral infections. A skin condition or skin lesion that can be treated according to the methods disclosed herein is any infected or irritated tissue that can be effectively treated by the application of cleansing, cooling and/or heating.

“Treating” a skin lesion or condition means to slow, halt or even reverse the development of skin lesions or conditions and to reduce the lesion's or condition's healing time. Therapeutic benefit can be achieved by eradication or amelioration of the underlying disorder being treated, e.g., eradication or amelioration of the acne, and/or eradication or amelioration of one or more of the physiological symptoms associated with the condition being treated, notwithstanding that the patient may still be afflicted with the underlying disorder. The terms “subject” and “animal subject” used herein typically refer to a human, but could also include other suitable animals.

The products used with the system can also be combined with topical gels or creams to improve treatment of skin lesions, by furthering the treatment effects of the device. For example, for acne, topical gel with benzoyl peroxide, antibiotics, anti-inflammatories, such as salicylic acid can be applied after treatment with the device. For improvement of fine wrinkles, topical lotions and gels can be applied after micro-abrasion which would have greater facility in being absorbed for maximal effect. Examples include glycolic acid, moisturizing emollients, and anti-oxidants such as green tea. For eczema, anti-inflammatories and pharmaceuticals can be applied directly afterwards for quicker and easier absorption into the skin for maximal effect.

The system can be used to improve drug delivery by applying drugs and creams to the skin before treatment. This may be in the form of a cleanser or micro-abrasion element to be worked into the skin surface by the device. It can also be micro-encapsulated pharmaceuticals or cosmetics that can be activated by the cold and mechanical abrasion of the device.

All publications and patent applications mentioned in this specification are herein incorporated by reference in their entireties. The above descriptions and illustrations are only by way of example and are not to be taken as limiting the invention in any manner. Numerous variations, changes, and substitutions can occur to those skilled in the art without departing from the invention. It should be understood that variations of the elements described herein may be employed. It is intended that the following claims define the scope of the invention and that methods and structures within the scope of these claims and their equivalents be covered thereby.

We claim:

1. A system for treatment of skin conditions comprising:
a pack comprising a flowable material for application to skin;
a hand piece configured to removably connect to the pack and apply the flowable material to the skin; and
a thermal device for changing the temperature of the pack.

2. The system of claim 1, wherein the thermal device comprises a cooling device.

3. The system of claim 2, wherein the thermal device comprises a heating device.

4. The system of claim 3, further comprising a control wherein the control is configured to activate the cooling device or the heating device.

5. The system of claim 4, wherein the heating device and the cooling device comprise the same thermoelectric element.

6. The system of claim 1, wherein the hand piece is powered to rotate and/or vibrate the pack.

7. The system of claim 1, wherein the thermal device comprises a heating device.

8. The system of claim 1, further comprising a control configured to alternate the thermal device between a heating function and a cooling function.

9. The system of claim 1, further comprising a container removably held in the pack, wherein at least some of the flowable material is stored in the removable container.

10. The system of claim 1, wherein the flowable material comprises a drug.

11. The system of claim 1, wherein the pack comprises an abrasive surface for applying to the skin.

12. The system of claim 1, wherein the hand piece comprises an abrasive surface for applying to the skin.

13. A system for applying personal care products:
a container containing a flowable material;
a dispensing cap that includes a thermal device to heat or cool the flowable material as dispensed.

14. A method of treating a skin condition comprising:
thermally altering a pack comprising a flowable material to a desired temperature;
grasping the pack with a hand piece; and
rubbing the skin with the pack to cleanse and rejuvenate the skin.

15. The method of claim 14, wherein thermally altering comprises cooling.

16. The method of claim 15, wherein thermally altering comprises heating.

17. The method of claim 14, wherein thermally altering comprises heating.

18. The method of claim 14, further comprising toggling between a heating function and a cooling function.

19. The method of claim 14, further comprising loading into the pack a container comprising at least some of the flowable material.

20. The method of claim 14, further comprising abrading the skin.
